(19) United States
(12) Patent Application Publication
(10) Pub. No.: US 2008/0008718 A1
(43) Pub. Date: Jan. 10, 2008

(54) Ornithobacterium Rhinotracheale Subunit Vaccines

(75) Inventors: Danielle Francisca Schuijffel, Langenboom (NL); Petrus Johnannes Maria Nuijten, Boxmeer (NL)

Correspondence Address: INTERVET INC.;PATENT DEPARTMENT, PO BOX 318, MILLSBORO, DE, 19966-0318, US

(73) Assignee: Intervet International B.V., Boxmeer (NL)

(21) Appl. No.: 10/588,992

(22) PCT Filed: Feb. 9, 2005
(86) PCT No.: PCT/EP05/50577

§ 371 Date: Aug. 10, 2006

(30) Foreign Application Priority Data
Feb. 11, 2004 (EP) 04075427.7

(51) Int. Cl. A61K 39/40; A61K 39/02; A61K 39/102; A61K 39/112; A61K 39/12; A61P 31/04; C07H 21/00; C12N 1/00; C07K 14/195; A61P 31/12; A61P 31/00; A61K 39/17; A61K 39/255; A61K 39/265

(52) U.S. Cl. 424/164.1; 424/211.1; 424/214.1; 424/229.1; 424/232.1; 424/233.1; 424/234.1; 424/255.1; 424/258.1; 424/264.1; 435/243; 530/350; 536/23.7

(57) ABSTRACT

The present invention relates to nucleic acids encoding Ornithobacterium rhinotracheale proteins, to DNA fragments, recombinant DNA molecules, live recombinant carriers and host cells comprising such nucleic acids, to Ornithobacterium rhinotracheale proteins, to antibodies against such proteins, to such proteins for use in vaccines, to the use of such proteins in the manufacturing of such vaccines, to vaccines comprising such nucleic acids, DNA fragments, recombinant DNA molecules, live recombinant carriers, host cells, proteins or antibodies against such proteins, and to methods for the preparation of such vaccines.

The present invention relates to nucleic acids encoding Ornithobacterium rhinotracheale proteins, to DNA fragments, recombinant DNA molecules, live recombinant carriers and host cells comprising such nucleic acids, to Ornithobacterium rhinotracheale proteins, to antibodies against such proteins, to such proteins for use in vaccines, to the use of such proteins in the manufacturing of such vaccines, to vaccines comprising such nucleic acids, DNA fragments, recombinant DNA molecules, live recombinant carriers, host cells, proteins or antibodies against such proteins, and to methods for the preparation of such vaccines.

Ornithobacterium rhinotracheale is a relatively recently discovered bacterium that is found more and more frequently in poultry farms, and in wild birds. Especially animals in commercial chicken farms, turkey farms and duck farms are frequently infected. In commercial poultry, infection is associated with respiratory diseases: airsacculitis and pneumonia are the most common features of infection with Ornithobacterium rhinotracheale. These signs can be induced by aerosol in intra-tracheal or intra-thoracic administration of the organism and are aggravated by other factors such as respiratory viruses, bacteria or suboptimal housing conditions. Osteitis, meningitis and joint-infections which can be induced by intravenous application have been associated with Ornithobacterium rhinotracheale. The infection can be transmitted horizontally, as well as vertically through eggs, which probably accounts for its rapid and worldwide spread. An extensive review of Ornithobacterium rhinotracheale has been given by van Empel, P. C. M. ad Hafez, H. M. in Avian Pathology 28:217-227 (1999). European Patent EP0.625.190 relates to both the Ornithobacterium rhinotracheale bacterium and to vaccines against Ornithobacterium rhinotracheale.

Serological research has revealed that Ornithobacterium rhinotracheale strains may have different serotypes, to a certain degree depending on the geographic origin of the strain and the host animal from which they were isolated. At this moment, eighteen different serotypes are found.

Therapeutic treatment of the disease can be difficult because acquired resistance against the regular antibiotics is very common within the genus. Moreover, there is an increasing reluctance against the use of antibiotics in food animals for both public health- and environmental reasons.

Vaccination offers an alternative for therapeutic treatment with antibiotics, but up till now, only vaccination with live attenuated vaccines and inactivated whole cell vaccines was possible.

The success of live attenuated vaccines specifically for Ornithobacterium rhinotracheale depends highly on the right balance between attenuation and triggering of the immune system. Inactivated whole cell vaccines are basically safe and therefore, from a safety point of view would seem the preferred type of vaccine.

Inactivated whole cell vaccines however need to be given in a higher dose compared to live attenuated vaccines. As a general rule, most of the proteins present in a bacterium play no role in the triggering of the immune system, i.e. they are not relevant immunogens. This means that, in the case of inactivated whole cell vaccines, in order to provide humans or animals with a sufficient level of relevant immunogens a lot of non-protective material is additionally and unavoidably administered. This is not a desirable situation.

The use of subunit vaccines could overcome this problem, and would therefore be highly preferred, but currently no immunogenic subunit vaccines are known in the art for combating Ornithobacterium rhinotracheale.

Moreover, although live attenuated vaccines and inactivated whole cell preparations are known to provide a certain level of cross-protection against all Ornithobacterium rhinotracheale strains, subunit vaccines might or might not induce cross-reactivity.

The present invention aims at providing for the first time vaccines that are based upon Ornithobacterium rhinotracheale subunits that do induce cross-reactivity.

This objective is reached by providing eight novel Ornithobacterium rhinotracheale proteins that surprisingly play an important role in triggering a protective immune response, and by providing vaccines comprising one or more of these novel immunogenic proteins.

Even more surprisingly, these eight novel proteins were found no only to induce a protective homologous immune response, but to also induce a protective cross-reactive immune response.

A homologous immune response is a response against strains of the same serotype, whereas a cross-reactive immune response is a response against both serologically homologous and heterologous strains.

The first novel protein, Or01, having a molecular weight of 59.8 kD is encoded by a nucleic acid having a nucleotide sequence as depicted in SEQ ID NO: 1.

It is well-known in the art, that many different nucleotide sequences can encode one and the same protein. This phenomenon is commonly known as wobble in the second and especially the third base of each triplet encoding an amino acid. This phenomenon can result in a heterology of about 20-30% for two nucleotide sequences still encoding the same protein. Therefore, two nucleic acids having a nucleotide sequence homology of about 80% can still encode one and the same protein.

Thus, one embodiment relates to a nucleic acid encoding a 59.8 kD Ornithobacterium rhinotracheale protein or a part of said nucleic acid that encodes an immunogenic fragment of said protein wherein said nucleic acid or said part thereof has at least 80% homology with the nucleic acid of the Ornithobacterium rhinotracheale protein gene as depicted in SEQ ID NO: 1.

The molecular weight of the protein (and the seven other proteins) is determined on the basis of the molecular weight of the amino acids as given in the amino acid sequence.

Preferably, a nucleic acid according to the invention encoding this 59.8 kD Ornithobacterium rhinotracheale protein or a part of that nucleic acid that encodes an immunogenic fragment of that protein has at least 85%, preferably 90%, more preferably 95% homology with the nucleic acid of the Ornithobacterium rhinotracheale protein gene as depicted in SEQ ID NO: 1.

Even more preferred is a homology level of 98%, 99% or even 100%.

The level of nucleotide homology can be determined with the computer program “BLAST 2 SEQUENCES” by selecting sub-program: “BLASTN” that can be found at www.ncbi.nlm.nih.gov/blast/b12seq/b12.html.

A reference for this program is Tatiana A. Tatusova, Thomas L. Madden FEMS Microbiol. Letters 174: 247-250 (1999). Parameters used are the default parameters:

Reward for a match: +1. Penalty for a mismatch: −2. Open gap: 5. Extension gap: 2. Gap x_dropoff: 50.

Another approach for deciding if a certain nucleic acid sequence is or is not a nucleic acid sequence according to the invention relates to the question if that certain nucleic acid sequence does hybridize under stringent conditions to the nucleotide sequence as depicted in SEQ ID NO: 1 (or in SEQ ID NO: 3, 5, 7, 9, 11, 13 or 15, see below).

If a nucleic acid sequence hybridizes under stringent conditions to the nucleotide sequence as depicted in SEQ ID NO: 1, or of course as depicted in SEQ ID NO: 3, 5, 7, 9, 11, 13 and 15, it is considered to be a nucleic acid sequence according to the invention.

The definition of stringent conditions follows from the formula of Meinkoth and Wahl (1984. Hybridization of nucleic acids immobilized on solid supports. Anal. Biochem. 138: 267-284.).

Tm=[81.5° C.+16.6(log M)+0.41(% GC)−0.61(% formamide)−500/L]−1° C./1% mismatch

In this formula, M is molarity of monovalent cations; % GC is the percentage of guanosine and cytosine nucleotides in the DNA; L is the length of the hybrid in base pairs.

Stringent conditions are those conditions under which nucleic acid sequences or fragments thereof still hybridize, if they have a mismatch of 20% at the most, preferably 10%, more preferably 8, 6, 5, 4, 3, 2, 1 or 0% in that order or preference, to the nucleic acid sequence as depicted in any of the SEQ ID NO: 1, 3, 5, 7, 9, 11, 13 or 15.

Another embodiment relates to a nucleic acid encoding a 58.2 kD Ornithobacterium rhinotracheale protein Or02, or a part of said nucleic acid that encodes an immunogenic fragment of said protein wherein said nucleic acid or said part thereof has at least 80% homology with the nucleic acid of the Ornithobacterium rhinotracheale protein gene as depicted in SEQ ID NO: 3.

Preferably, a nucleic acid according to the invention encoding this 58.2 kD Ornithobacterium rhinotracheale protein or a part of that nucleic acid that encodes an immunogenic fragment of that protein has at least 85%, preferably 90%, more preferably 95% homology with the nucleic acid of the Ornithobacterium rhinotracheale protein gene as depicted in SEQ ID NO: 3.

Even more preferred is a homology level of 98%, 99% or even 100%.

Still another embodiment relates to a nucleic acid encoding a 46.0 kD Ornithobacterium rhinotracheale protein Or03 or a part of said nucleic acid that encodes an immunogenic fragment of said protein wherein said nucleic acid or said part thereof has at least 80% homology with the nucleic acid of the Ornithobacterium rhinotracheale protein gene as depicted in SEQ ID NO: 5.

Preferably, a nucleic acid according to the invention encoding this 46.0 kD Ornithobacterium rhinotracheale protein or a part of that nucleic acid that encodes an immunogenic fragment of that protein has at least 85%, preferably 90%, more preferably 95% homology with the nucleic acid of the Ornithobacterium rhinotracheale protein gene as depicted in SEQ ID NO: 5.

Even more preferred is a homology level of 98%, 99% or even 100%.

Again another embodiment relates to a nucleic acid encoding a 37.2 kD Ornithobacterium rhinotracheale protein Or04 or a part of said nucleic acid that encodes an immunogenic fragment of said protein wherein said nucleic acid or said part thereof has at least 80% homology with the nucleic acid of the Ornithobacterium rhinotracheale protein gene as depicted in SEQ ID NO: 7.

Preferably, a nucleic acid according to the invention encoding this 37.2 kD Ornithobacterium rhinotracheale protein or a part of that nucleic acid that encodes an immunogenic fragment of that protein has at least 85%, preferably 90%, more preferably 95% homology with the nucleic acid of the Ornithobacterium rhinotracheale protein gene as depicted in SEQ ID NO: 7.

Even more preferred is a homology level of 98%, 99% or even 100%.

Another embodiment relates to a nucleic acid encoding a 45.6 kD Ornithobacterium rhinotracheale protein Or11 or a part of said nucleic acid that encodes an immunogenic fragment of said protein wherein said nucleic acid or said part thereof has at least 80% homology with the nucleic acid of the Ornithobacterium rhinotracheale protein gene as depicted in SEQ ID NO: 9.

Preferably, a nucleic acid according to the invention encoding this 45.6 kD Ornithobacterium rhinotracheale protein or a part of that nucleic acid that encodes an immunogenic fragment of that protein has at least 85%, preferably 90%, more preferably 95% homology with the nucleic acid of the Ornithobacterium rhinotracheale protein gene as depicted in SEQ ID NO: 9.

Even more preferred is a homology level of 98%, 99% or even 100%.

Again another embodiment relates to a nucleic acid encoding a 42.2 kD Ornithobacterium rhinotracheale protein Or77 or a part of said nucleic acid that encodes an immunogenic fragment of said protein wherein said nucleic acid or said part thereof has at least 80% homology with the nucleic acid of the Ornithobacterium rhinotracheale protein gene as depicted in SEQ ID NO: 11.

Preferably, a nucleic acid according to the invention encoding this 42.2 kD Ornithobacterium rhinotracheale protein or a part of that nucleic acid that encodes an immunogenic fragment of that protein has at least 85%, preferably 90%, more preferably 95% homology with the nucleic acid of the Ornithobacterium rhinotracheale protein gene as depicted in SEQ ID NO: 11.

Even more preferred is a homology level of 98%, 99% or even 100%.

Also another embodiment relates to a nucleic acid encoding a 34.0 kD Ornithobacterium rhinotracheale protein Or98A or a part of said nucleic acid that encodes an immunogenic fragment of said protein wherein said nucleic acid or said part thereof has at least 80% homology with the nucleic acid of the Ornithobacterium rhinotracheale protein gene as depicted in SEQ ID NO: 13.

Preferably, a nucleic acid according to the invention encoding this 34.0 kD Ornithobacterium rhinotracheale protein or a part of that nucleic acid that encodes an immunogenic fragment of that protein has at least 85%, preferably 90%, more preferably 95% homology with the nucleic acid of the Ornithobacterium rhinotracheale protein gene as depicted in SEQ ID NO: 13.

Even more preferred is a homology level of 98%, 99% or even 100%.

Another embodiment relates to a nucleic acid encoding a 32.9 kD Ornithobacterium rhinotracheale protein Or98B or a part of said nucleic acid that encodes an immunogenic fragment of said protein wherein said nucleic acid or said part thereof has at least 80% homology with the nucleic acid of the Ornithobacterium rhinotracheale protein gene as depicted in SEQ ID NO: 15.

Preferably, a nucleic acid according to the invention encoding this 32.9 kD Ornithobacterium rhinotracheale protein or a part of that nucleic acid that encodes an immunogenic fragment of that protein has at least 85%, preferably 90%, more preferably 95% homology with the nucleic acid of the Ornithobacterium rhinotracheale protein gene as depicted in SEQ ID NO: 15.

Even more preferred is a homology level of 98%, 99% or even 100%.

Nucleotide sequences that are complementary to the sequence depicted in SEQ ID NO 1, 3, 5, 7, 9, 11, 13 or 15 or nucleotide sequences that comprise tandem arrays of the sequences according to the invention are also within the scope of the invention.

Since the present invention discloses nucleic acids encoding 8 novel Ornithobacterium rhinotracheale proteins, it is now for the first time possible to obtain these proteins in significant quantities. This can e.g. be done by using expression systems to express the whole or parts of a gene encoding the protein or an immunogenic fragment thereof.

Therefore, in a preferred form of this embodiment, the invention relates to DNA fragments comprising a nucleic acid according to the invention. A DNA fragment is a stretch of nucleotides that functions as a carrier for a nucleic acid according to the invention. Such DNA fragments can e.g. be plasmids, into which a nucleic acid according to the invention is cloned. Such DNA fragments are e.g. useful for enhancing the amount of DNA for use as a primer and for expression of a nucleic acid according to the invention, as described below.

An essential requirement for the expression of the nucleic acid is an adequate promoter functionally linked to the nucleic acid, so that the nucleic acid is under the control of the promoter. It is obvious to those skilled in the art that the choice of a promoter extends to any eukaryotic, prokaryotic or viral promoter capable of directing gene transcription in cells used as host cells for protein expression.

Therefore, a more preferred form of this embodiment relates to a recombinant DNA molecule comprising a DNA fragment and/or a nucleic acid according to the invention wherein the nucleic acid according to the invention is placed under the control of a functionally linked promoter. This can be obtained by means of e.g. standard molecular biology techniques. (Maniatis/Sambrook (Sambrook, J. Molecular cloning: a laboratory manual, 1989. ISBN 0-87969-309-6).

Functionally linked promoters are promoters that are capable of controlling the transcription of the nucleic acids to which they are linked.

Such a promoter can be the native promoter of the novel gene, i.e. the promoter that is involved in the transcription of the nucleic acid encoding a protein according to the invention, or another promoter of Ornithobacterium rhinotracheale, provided that that promoter is functional in the cell used for expression. It can also be a heterologous promoter. When the host cells are bacteria, useful expression control sequences which may be used include the Trp promoter and operator (Goeddel, et al., Nucl. Acids Res., 8, 4057, 1980); the lac promoter and operator (Chang, et al., Nature, 275, 615, 1978); the outer membrane protein promoter (Nakamura, K. and Inouge, M., EMBO J., 1, 771-775, 1982); the bacteriophage lambda promoters and operators (Remaut, E. et al., Nucl. Acids Res., 11, 4677-4688, 1983); the α-amylase (B. subtilis) promoter and operator, termination sequences and other expression enhancement and control sequences compatible with the selected host cell.

When the host cell is yeast, useful expression control sequences include, e.g., α-mating factor. For insect cells the polyhedrin or p10 promoters of baculoviruses can be used (Smith, G. E. et al., Mol. Cell. Biol. 3, 2156-65, 1983). When the host cell is of vertebrate origin illustrative useful expression control sequences include the (human) cytomegalovirus immediate early promoter (Seed, B. et al., Nature 329, 840-842, 1987; Fynan, E. F. et al., PNAS 90, 11478-11482, 1993; Ulmer, J. B. et al., Science 259, 1745-1748, 1993), Rous sarcoma virus LTR (RSV, Gorman, C. M. et al., PNAS 79, 6777-6781, 1982; Fynan et al., supra; Ulmer et al., supra), the MPSV LTR (Stacey et al., J. Virology 50, 725-732, 1984), SV40 immediate early promoter (Sprague J. et al., J. Virology 45, 773, 1983), the SV-40 promoter (Berman, P. W. et al., Science, 222, 524-527, 1983), the metallothionein promoter (Brinster, R. L. et al., Nature 296, 39-42, 1982), the heat shock promoter (Voellmy et al., Proc. Natl. Acad. Sci. USA, 82, 4949-53, 1985), the major late promoter of Ad2 and the β-actin promoter (Tang et al., Nature 356, 152-154, 1992). The regulatory sequences may also include terminator and poly-adenylation sequences. Amongst the sequences that can be used are the well known bovine growth hormone poly-adenylation sequence, the SV40 poly-adenylation sequence, the human cytomegalovirus (hCMV) terminator and poly-adenylation sequences.

Bacterial, yeast, fungal, insect and vertebrate cell expression systems are very frequently used systems. Such systems are well-known in the art and generally available, e.g. commercially through Clontech Laboratories, Inc. 4030 Fabian Way, Palo Alto, Calif. 94303-4607, USA. Next to these expression systems, parasite-based expression systems are attractive expression systems. Such systems are e.g. described in the French Patent Application with Publication number 2 714 074, and in U.S. NTIS Publication No U.S. Ser. No. 08/043,109 (Hoffman, S, and Rogers, W.: Public. Date 1 Dec. 1993).

An even more preferred form of this embodiment of the invention relates to Live Recombinant Carriers (LRCs) comprising a nucleic acid encoding an Ornithobacterium rhinotracheale protein or an immunogenic fragment thereof according to the invention, a DNA fragment according to the invention or a recombinant DNA molecule according to the invention. These LRCs are micro-organisms or viruses in which additional genetic information, in this case a nucleic acid encoding an Ornithobacterium rhinotracheale protein or an immunogenic fragment thereof, a DNA fragment or a recombinant DNA molecule according to the invention has been cloned. Chickens infected with such LRCs will produce an immunological response not only against the immunogens of the carrier, but also against the immunogenic parts of the protein(s) for which the genetic code is additionally cloned into the LRC, e.g. an Ornithobacterium rhinotracheale protein gene according to the invention.

As an example of bacterial LRCs, attenuated Salmonella strains known in the art can very attractively be used.

Also, live recombinant carrier parasites have i.a. been described by Vermeulen, A. N. (Int. Journ. Parasitol. 28: 1121-1130 (1998)).

Furthermore, LRC viruses may be used as a way of transporting the nucleic acid into a target cell. Live recombinant carrier viruses are also called vector viruses. Viruses often used as vectors are Vaccinia viruses (Panicali et al; Proc. Natl. Acad. Sci. USA, 79: 4927 (1982), Herpesviruses (E.P.A. 0473210A2), and Retroviruses (Valerio, D. et al; in Baum, S. J., Dicke, K. A., Lotzova, E. and Pluznik, D. H. (Eds.), Experimental Haematology today—1988. Springer Verlag, New York: pp. 92-99 (1989)).

Viruses known and used in the art as very suitable vector viruses specifically in poultry are Fowlpox virus, Marek's serotype 3 virus, Herpes virus of Turkey, Semlikl Forest virus and Newcastle Disease virus.

Live Recombinant Carriers are also known in the art as “live vectors”, or shortly “vectors”. Vaccines based upon a Live Recombinant Carrier are therefore also known in the art as vector vaccines.

The technique of in vivo homologous recombination, well-known in the art, can be used to introduce a recombinant nucleic acid into the genome of a bacterium, parasite or virus of choice, capable of inducing expression of the inserted nucleic acid according to the invention in the host animal.

Finally another form of this embodiment of the invention relates to a host cell comprising a nucleic acid encoding a protein according to the invention, a DNA fragment comprising such a nucleic acid or a recombinant DNA molecule comprising such a nucleic acid under the control of a functionally linked promoter. This form also relates to a host cell containing a live recombinant carrier comprising a nucleic acid molecule encoding an Ornithobacterium rhinotracheale protein or an immunogenic fragment thereof according to the invention. A host cell may be a cell of bacterial origin, e.g. Escherichia coli, Bacillus subtilis and Lactobacillus species, in combination with bacteria-based plasmids as pBR322, or bacterial expression vectors as pGEX, or with bacteriophages. The host cell may also be of eukaryotic origin, e.g. yeast-cells in combination with yeast-specific vector molecules, or higher eukaryotic cells like insect cells (Luckow et al; Bio-technology 6: 47-55 (1988)) in combination with vectors or recombinant baculoviruses, plant cells in combination with e.g. Ti-plasmid based vectors or plant viral vectors (Barton, K. A. et al; Cell 32: 1033 (1983), mammalian cells like Hela cells, Chinese Hamster Ovary cells (CHO) or Crandell Feline Kidney-cells, also with appropriate vectors or recombinant viruses.

Another embodiment of the invention relates to an Ornithobacterium rhinotracheale protein and to immunogenic fragments thereof according to the invention.

The concept of immunogenic fragments will be defined below.

One form of this embodiment relates to a 59.8 kD Ornithobacterium rhinotracheale protein and to immunogenic fragments thereof, having an amino acid sequence homology of at least 80% with the amino acid sequence as depicted in SEQ ID NO: 2.

In a preferred form, the embodiment relates to such Ornithobacterium rhinotracheale proteins and immunogenic fragments thereof, that have a sequence homology of at least 85%, preferably 90%, more preferably 95% homology to the amino acid sequence as depicted in SEQ ID NO: 2.

Even more preferred is a homology level of 98%, 99% or even 100%.

The level of protein homology can be determined with the computer program “BLAST 2 SEQUENCES” by selecting sub-program: “BLASTP”, that can be found at www.ncbi.nlm.nih.gov/blast/b12seq/b12.html.

A reference for this program is Tatiana A. Tatusova, Thomas L. Madden FEMS Microbiol. Letters 174: 247-250 (1999). Matrix used: “blosum62”. Parameters used are the default parameters:

Open gap: 11. Extension gap: 1. Gap x_dropoff: 50.

Another form of this embodiment relates to a 58.2 kD) Ornithobacterium rhinotracheale protein and to immunogenic fragments thereof, having an amino acid sequence homology of at least 80% with the amino acid sequence as depicted in SEQ ID NO: 4.

In a preferred form, the embodiment relates to such Ornithobacterium rhinotracheale proteins and immunogenic fragments thereof, that have a sequence homology of at least 85%, preferably 90%, more preferably 95% homology to the amino acid sequence as depicted in SEQ ID NO: 4.

Even more preferred is a homology level of 98%, 99% or even 100%.

Still another form of this embodiment relates to a 46.0 kD Ornithobacterium rhinotracheale protein and to immunogenic fragments thereof, having an amino acid sequence homology of at least 80% with the amino acid sequence as depicted in SEQ ID NO: 6.

In a preferred form, the embodiment relates to such Ornithobacterium rhinotracheale proteins and immunogenic fragments thereof, that have a sequence homology of at least 85%, preferably 90%, more preferably 95% homology to the amino acid sequence as depicted in SEQ ID NO: 6.

Even more preferred is a homology level of 98%, 99% or even 100%.

Again another form of this embodiment relates to a 37.2 kD Ornithobacterium rhinotracheale protein and to immunogenic fragments thereof, having an amino acid sequence homology of at least 80% with the amino acid sequence as depicted in SEQ ID NO: 8.

In a preferred form, the embodiment relates to such Ornithobacterium rhinotracheale proteins and immunogenic fragments thereof, that have a sequence homology of at least 85%, preferably 90%, more preferably 95% homology to the amino acid sequence as depicted in SEQ ID NO: 8.

Even more preferred is a homology level of 98%, 99% or even 100%.

Still another form of this embodiment relates to a 45.6 kD Ornithobacterium rhinotracheale protein and to immunogenic fragments thereof, having an amino acid sequence homology of at least 80% with the amino acid sequence as depicted in SEQ ID NO: 10.

In a preferred form, the embodiment relates to such Ornithobacterium rhinotracheale proteins and immunogenic fragments thereof, that have a sequence homology of at least 85%, preferably 90%, more preferably 95% homology to the amino acid sequence as depicted in SEQ ID NO: 10.

Even more preferred is a homology level of 98%, 99% or even 100%.

One other form of this embodiment relates to a 42.2 kD Ornithobacterium rhinotracheale protein and to immunogenic fragments thereof, having an amino acid sequence homology of at least 80% with the amino acid sequence as depicted in SEQ ID NO: 12.

In a preferred form, the embodiment relates to such Ornithobacterium rhinotracheale proteins and immunogenic fragments thereof, that have a sequence homology of at least 85%, preferably 90%, more preferably 95% homology to the amino acid sequence as depicted in SEQ ID NO: 12.

Even more preferred is a homology level of 98%, 99% or even 100%.

And again another form of this embodiment relates to a 34.0 kD Ornithobacterium rhinotracheale protein and to immunogenic fragments thereof, having an amino acid sequence homology of at least 80% with the amino acid sequence as depicted in SEQ ID NO: 14.

In a preferred form, the embodiment relates to such Ornithobacterium rhinotracheale proteins and immunogenic fragments thereof, that have a sequence homology of at least 85%, preferably 90%, more preferably 95% homology to the amino acid sequence as depicted in SEQ ID NO: 14.

Even more preferred is a homology level of 98%, 99% or even 100%.

Finally another form of this embodiment relates to a 32.9 kD Ornithobacterium rhinotracheale protein and to immunogenic fragments thereof, having an amino acid sequence homology of at least 80% with the amino acid sequence as depicted in SEQ ID NO: 16.

In a preferred form, the embodiment relates to such Ornithobacterium rhinotracheale proteins and immunogenic fragments thereof, that have a sequence homology of at least 85%, preferably 90%, more preferably 95% homology to the amino acid sequence as depicted in SEQ ID NO: 16.

Even more preferred is a homology level of 98%, 99% or even 100%.

Another form of this embodiment relates to such Ornithobacterium rhinotracheale proteins and immunogenic fragments of said proteins according to the invention, wherein the proteins and immunogenic fragments thereof are encoded by a nucleic acid according to the invention.

It will be understood that, for the particular proteins embraced herein, natural variations can exist between individual Ornithobacterium rhinotracheale strains. These variations may be demonstrated by (an) amino acid difference(s) in the overall sequence or by deletions, substitutions, insertions, inversions or additions of (an) amino acid(s) in said sequence. Amino acid substitutions which do not essentially alter biological and immunological activities, have been described, e.g. by Neurath et al in “The Proteins” Academic Press New York (1979). Amino acid replacements between related amino acids or replacements which have occurred frequently in evolution are, inter alia, Ser/Ala, Ser/Gly, Asp/Gly, Asp/Asn, Ile/Val (see Dayhof, M. D., Atlas of protein sequence and structure, Nat. Biomed. Res. Found., Washington D.C., 1978, vol. 5, suppl. 3). Other amino acid substitutions include Asp/Glu, Thr/Ser, Ala/Gly, Ala/Thr, Ser/Asn, Ala/Val, Thr/Phe, Ala/Pro, Lys/Arg, Leu/Ile, Leu/Val and Ala/Glu. Based on this information, Lipman and Pearson developed a method for rapid and sensitive protein comparison (Science, 227, 1435-1441, 1985) and determining the functional similarity between homologous proteins. Such amino acid substitutions of the exemplary embodiments of this invention, as well as variations having deletions and/or insertions are within the scope of the invention as long as the resulting proteins retain their immune reactivity.

This explains why Ornithobacterium rhinotracheale proteins according to the invention, when isolated from different field isolates, may have homology levels as low as about 80%, while still representing the same protein with the same immunological characteristics. Those variations in the amino acid sequence of a certain protein according to the invention that still provide a protein capable of inducing an immune response against infection with Ornithobacterium rhinotracheale or at least against the clinical manifestations of the infection are considered as “not essentially influencing the immunogenicity”.

When a protein is used for e.g. vaccination purposes or for raising antibodies, it is however not necessary to use the whole protein. It is also possible to use a fragment of that protein that is capable, as such or coupled to a carrier such as e.g. KLH, of inducing an immune response against that protein, a so-called immunogenic fragment. An “immunogenic fragment” is understood to be a fragment of the full-length protein that still has retained its capability to induce an immune response in a vertebrate host, e.g. comprises a B- or T-cell epitope.

Shortly, an immunogenic fragment is a fragment that is capable of inducing an antigenic response against an Ornithobacterium rhinotracheale protein according to the invention. At this moment, a variety of techniques is available to easily identify DNA fragments encoding antigenic fragments (determinants). The method described by Geysen et al (Patent Application WO 84/03564, Patent Application WO 86/06487, U.S. Pat. No. 4,833,092, Proc. Natl. Acad. Sci. 81: 3998-4002 (1984), J. 1 mm. Meth. 102, 259-274 (1987), the so-called PEPSCAN method is an easy to perform, quick and well-established method for the detection of epitopes; the immunologically important regions of the protein. The method is used worldwide and as such well-known to man skilled in the art. This (empirical) method is especially suitable for the detection of B-cell epitopes. Also, given the sequence of the gene encoding any protein, computer algorithms are able to designate specific protein fragments as the immunologically important epitopes on the basis of their sequential and/or structural agreement with epitopes that are now known. The determination of these regions is based on a combination of the hydrophilicity criteria according to Hopp and Woods (Proc. Natl. Acad. Sci. 78: 38248-3828 (1981)), and the secondary structure aspects according to Chou and Fasman (Advances in Enzymology 47: 45-148 (1987) and U.S. Pat. No. 4,554,101). T-cell epitopes can likewise be predicted from the sequence by computer with the aid of Berzofsky's amphiphilicity criterion (Science 235, 1059-1062 (1987) and U.S. patent application NTIS U.S. Ser. No. 07/005,885). A condensed overview is found in: Shan Lu on common principles: Tibtech 9: 238-242 (1991), Good et al on Malaria epitopes; Science 235: 1059-1062 (1987), Lu for a review; Vaccine 10: 3-7 (1992), Berzofsky for HIV-epitopes; The FASEB Journal 5:2412-2418 (1991). An immunogenic fragment usually has a minimal length of 8 amino acids, preferably more then 8, such as 9, 10, 12, 15 or even 20 amino acids. The nucleic acids encoding such a fragment therefore have a length of at least 24, but preferably 27, 30, 36, 45 or even 60 nucleic acids.

Therefore, one form of still another embodiment of the invention relates to vaccines for combating Ornithobacterium rhinotracheale infection, that comprise an Ornithobacterium rhinotracheale protein or immunogenic fragments thereof, according to the invention as described above together with a pharmaceutically acceptable carrier.

Still another embodiment of the present invention relates to an Ornithobacterium rhinotracheale protein according to the invention or immunogenic fragments thereof for use in a vaccine.

Still another embodiment of the present invention relates to the use of a nucleic acid, a DNA fragment, a recombinant DNA molecule, a live recombinant carrier, a host cell or a protein or an immunogenic fragment thereof according to the invention for the manufacturing of a vaccine for combating Ornithobacterium rhinotracheale infection.

One way of making a vaccine according to the invention is by growing the bacteria, followed by biochemical purification of an Ornithobacterium rhinotracheale protein or an immunogenic fragment thereof, from the bacterium. This is however a very time-consuming way of making the vaccine.

It is therefore much more convenient to use the expression products of the gene encoding an Ornithobacterium rhinotracheale protein or immunogenic fragments thereof in vaccines. This is possible for the first time now because the nucleic acids encoding the Ornithobacterium rhinotracheale proteins are provided in the present invention.

Vaccines based upon the expression products of these genes can easily be made by admixing the protein according to the invention or immunogenic fragments thereof according to the invention with a pharmaceutically acceptable carrier as described below.

Alternatively, a vaccine according to the invention can comprise live recombinant carriers as described above, capable of expressing the protein according to the invention or immunogenic fragments thereof. Such vaccines, e.g. based upon a Salmonella carrier or a viral carrier e.g. a Herpesvirus vector have the advantage over subunit vaccines that they better mimic the natural way of infection of Ornithobacterium rhinotracheale. Moreover, their self-propagation is an advantage since only low amounts of the recombinant carrier are necessary for immunization.

Vaccines can also be based upon host cells as described above, that comprise the protein or immunogenic fragments thereof according to the invention.

All vaccines described above contribute to active vaccination, i.e. they trigger the host's defense system.

Alternatively, antibodies can be raised in e.g. rabbits or can be obtained from antibody-producing cell lines as described below. Such antibodies can then be administered to the chicken. This method of vaccination, passive vaccination, is the vaccination of choice when an animal is already infected, and there is no time to allow the natural immune response to be triggered. It is also the preferred method for vaccinating animals that are prone to sudden high infection pressure. The administered antibodies against the protein according to the invention or immunogenic fragments thereof can in these cases bind directly to Ornithobacterium rhinotracheale. This has the advantage that it decreases or stops Ornithobacterium rhinotracheale multiplication.

Therefore, one other form of this embodiment of the invention relates to a vaccine for combating Ornithobacterium rhinotracheale infection that comprises antibodies against a Ornithobacterium rhinotracheale protein according to the invention or an immunogenic fragment of that protein, and a pharmaceutically acceptable carrier.

Still another embodiment of this invention relates to antibodies against a Ornithobacterium rhinotracheale protein according to the invention or an immunogenic fragment of that protein.

Methods for large-scale production of antibodies according to the invention are also known in the art Such methods rely on the cloning of (fragments of) the genetic information encoding the protein according to the invention in a filamentous phage for phage display. Such techniques are described i.a. at the “Antibody Engineering Page” under “filamentous phage display” at http://aximt1.imt.uni-marburg.de/˜rek/aepphage.html., and in review papers by Cortese, R. et al., (1994) in Trends Biotechn. 12: 262-267, by Clackson, T. & Wells, J. A. (1994) in Trends Biotechn. 12: 173-183, by Marks, J. D. et al., (1992) in J. Biol. Chem. 267: 16007-16010, by Winter, G. et al., (1994) in Annu. Rev. Immunol. 12: 433-455, and by Little, M. et al., (1994) Biotechn. Adv. 12: 539-555. The phages are subsequently used to screen camelid expression libraries expressing camelid heavy chain antibodies. (Muyldermans, S, and Lauwereys, M., Journ. Molec. Recogn. 12: 131-140 (1999) and Ghahroudi, M. A. et al., FEBS Letters 414: 512-526 (1997)). Cells from the library that express the desired antibodies can be replicated and subsequently be used for large scale expression of antibodies.

Still another embodiment relates to a method for the preparation of a vaccine according to the invention that comprises the admixing of antibodies according to the invention and a pharmaceutically acceptable carrier.

An alternative and efficient way of vaccination is direct vaccination with DNA encoding the relevant antigen. Direct vaccination with DNA encoding proteins has been successful for many different proteins. (As reviewed in e.g. Donnelly et al., The Immunologist 2: 20-26 (1993)). This way of vaccination is also attractive for the vaccination of chickens against Ornithobacterium rhinotracheale infection.

Therefore, still other forms of this embodiment of the invention relate to vaccines comprising nucleic acids encoding a protein according to the invention or immunogenic fragments thereof, comprising DNA fragments that comprise such nucleic acids or comprising recombinant DNA molecules according to the invention, and a pharmaceutically acceptable carrier.

Examples of DNA plasmids that are suitable for use in a DNA vaccine according to the invention are conventional cloning or expression plasmids for bacterial, eukaryotic and yeast host cells, many of said plasmids being commercially available. Well-known examples of such plasmids are pBR322 and pcDNA3 (Invitrogen). The DNA fragments or recombinant DNA molecules according to the invention should be able to induce protein expression of the nucleotide sequences. The DNA fragments or recombinant DNA molecules may comprise one or more nucleotide sequences according to the invention. In addition, the DNA fragments or recombinant DNA molecules may comprise other nucleotide sequences such as the immune-stimulating oligonucleotides having unmethylated CpG di-nucleotides, or nucleotide sequences that code for other antigenic proteins or adjuvating cytokines.

The nucleotide sequence according to the present invention or the DNA plasmid comprising a nucleotide sequence according to the present invention, preferably operably linked to a transcriptional regulatory sequence, to be used in the vaccine according to the invention can be naked or can be packaged in a delivery system. Suitable delivery systems are lipid vesicles, iscoms, dendromers, niosomes, polysaccharide matrices and the like, (see further below) all well-known in the art. Also very suitable as delivery system are attenuated live bacteria such as Salmonella species, and attenuated live viruses such as Herpesvirus vectors, as mentioned above.

DNA vaccines can e.g. easily be administered through intradermal application such as by using a needle-less injector. This way of administration delivers the DNA directly into the cells of the animal to be vaccinated. Amounts of DNA in the range between 10 pg and 1000 μg provide good results. Preferably, amounts in the microgram range between 1 and 100 μg are used.

In a further embodiment, the vaccine according to the present invention comprises one or more additional antigens derived from a virus or micro-organism pathogenic to poultry, an antibody against such an antigen or genetic information encoding said antigen.

Of course, such antigens can be e.g. other Ornithobacterium rhinotracheale antigens. It is beneficial to combine, in one vaccine, two or more of the proteins or immunogenic fragments thereof according to the invention, antibodies against such proteins or immunogenic fragments thereof, or genetic information encoding such proteins or immunogenic fragments thereof.

Next to this, it is beneficial to include in a vaccine according to the invention, antigens derived from another micro-organism or a virus pathogenic to poultry, an antibody against such an antigen or genetic information encoding said antigen.

Preferably, the virus or micro-organism is selected from the group consisting of Fowlpox virus, Infectious Bronchitis virus, Infectious Bursal Disease (Gumboro), Marek's Disease Virus, Chicken Anaemia agent, Avian Reovirus, Mycoplasma gallisepticum, Turkey Rhinotracheitis virus, Haemophilus paragallinarum (Coryza), Chicken Poxvirus, Avian Encephalomyelitisvirus, Duck Plague virus, Newcastle Disease virus, Egg prop syndrome virus, Infectious Laryngotracheitis virus, Herpes Virus of Turkeys, Eimeria species, Ornithobacterium rhinotracheale, Pasteurella multocida, Mycoplasma synoviae, Salmonella species and E. coli.

Vaccines based upon the Ornithobacterium rhinotracheale proteins according to the invention are also very suitable as marker vaccines. A marker vaccine is a vaccine that allows to discriminate between vaccinated and field-infected chickens e.g. on the basis of a characteristic antibody panel, different from the antibody panel induced by wild type infection. A different antibody panel is induced e.g. when an immunogenic protein present on a wild type bacterium is not present in a vaccine: the host will then not make antibodies against that protein after vaccination. Thus, a vaccine based upon an Ornithobacterium rhinotracheale protein according to the invention would only induce antibodies against that protein, whereas a vaccine based upon a live wild-type, live attenuated or inactivated whole Ornithobacterium rhinotracheale would induce antibodies against all or most of the bacterial proteins.

A simple ELISA test, having wells comprising one protein according to the invention and wells comprising another protein according to the invention suffices to test serum from chickens and to tell if the chickens are either vaccinated with a subunit vaccine according to the invention or suffered from Ornithobacterium rhinotracheale field infection; chickens vaccinated with a vaccine comprising one protein according to the invention would not have antibodies against another protein according to the invention. Chickens that have encountered a field infection with Ornithobacterium rhinotracheale would however have antibodies against all immunogenic Ornithobacterium rhinotracheale proteins and thus also against another protein according to the invention.

All vaccines according to the present invention comprise a pharmaceutically acceptable carrier. A pharmaceutically acceptable carrier can be e.g. sterile water or a sterile physiological salt solution. In a more complex form the carrier can e.g. be a buffer.

Methods for the preparation of a vaccine comprise the admixing of a protein or an immunogenic fragment thereof, according to the invention and/or antibodies against that protein or an immunogenic fragment thereof, and/or a nucleic acid and/or a DNA fragment, a recombinant DNA molecule, a live recombinant carrier or host cell according to the invention, and a pharmaceutically acceptable carrier.

Vaccines according to the present invention may in a preferred presentation also contain an immunostimulatory substance, a so-called adjuvant. Adjuvants in general comprise substances that boost the immune response of the host in a non-specific manner. A number of different adjuvants are known in the art. Examples of adjuvants frequently used in chicken vaccines are muramyldipeptides, lipopolysaccharides, several glucans and glycans and Carbopol® (a homopolymer).

The vaccine may also comprise a so-called “vehicle”. A vehicle is a compound to which the protein adheres, without being covalently bound to it. Such vehicles are i.a. bio-microcapsules, micro-alginates, liposomes and macrosols, all known in the art.

A special form of such a vehicle, in which the antigen is partially embedded in the vehicle, is the so-called ISCOM (EP 109.942, EP 180.564, EP 242.380)

In addition, the vaccine may comprise one or more suitable surface-active compounds or emulsifiers, e.g. Span or Tween.

Often, the vaccine is mixed with stabilisers, e.g. to protect degradation-prone proteins from being degraded, to enhance the shelf-life of the vaccine, or to improve freeze-drying efficiency. Useful stabilisers are i.a. SPGA (Bovarnik et al; J. Bacteriology 59: 509 (1950)), carbohydrates e.g. sorbitol, mannitol, trehalose, starch, sucrose, dextran or glucose, proteins such as albumin or casein or degradation products thereof, and buffers, such as alkali metal phosphates.

In addition, the vaccine may be suspended in a physiologically acceptable diluent. It goes without saying, that other ways of adjuvating, adding vehicle compounds or diluents, emulsifying or stabilising a protein are also embodied in the present invention.

Vaccines according to the invention that are based upon the protein according to the invention or immunogenic fragments thereof can very suitably be administered in amounts ranging between 1 and 100 micrograms of protein per animal, although smaller doses can in principle be used. A dose exceeding 100 micrograms will, although immunologically very suitable, be less attractive for commercial reasons.

Vaccines based upon live attenuated recombinant carriers, such as the LRC-viruses and bacteria described above can be administered in much lower doses, because they multiply themselves during the infection. Therefore, very suitable amounts would range between 10³ and 10⁹ CFU/PFU for respectively bacteria/viruses.

Vaccines according to the invention can be administered e.g. intradermally, subcutaneously, intramuscularly, intraperitoneally, intravenously, or at mucosal surfaces such as orally or intranasally.

Live recombinant carrier vaccines or vector vaccines can most efficiently be administered by spraying, by aerosol or by drinking water administration.

### EXAMPLES

### Example 1

### Library Construction, Sera and Screening

For the construction of an expression library of Ornithobacterium rhinotracheale serotype G strain O-95029 nr.16279, genomic DNA was isolated from cells grown in Todd Hewitt broth (THB) for 24 hours at 37° C. on a 100 rpm shaker, according to the method described in Maniatis/Sambrook (Sambrook, J. et al. Molecular cloning: a laboratory manual. ISBN 0-87969-309-6). DNA fragments of 1-4 kb were obtained by restriction enzyme digestion and ligated into λTriplEx vector arms (Clontech, Palo Alto, Calif., USA). Subsequent packaging was performed using the Stratagene (La Jolla, Calif., USA) in vitro packaging extract. Escherichia coli XL1 Blue cells, grown in Luria Bertani (LB) broth supplemented with 10 mM MgSO₄ and 0.2% maltose, were used for transfection. The complexity of the constructed expression library was tested 6.9 and it contained 97% recombinants.

The Ornithobacterium rhinotracheale serotype G expression library was screened with polyclonal antisera directed against whole live organisms of several Ornithobacterium rhinotracheale serotypes. Sera were collected from broiler chickens that were vaccinated by aerosol spraying with live Ornithobacterium rhinotracheale bacteria of serotype B (strain GGD 1261), serotype G (strain O-95029 nr.16279) or serotype M (strain TOP 98036 4500) at two weeks of age. Three weeks later the chickens were intravenously challenged with Ornithobacterium rhinotracheale serotype A (strain B3263/91). Sera were collected one week after challenge. All vaccinated birds showed reduced pathology (ranging from 10% to 60%) in comparison to unvaccinated control birds. Before use in expression library screening, the antisera were adsorbed with Escherichia coli XL1 Blue cell lysate as described in Maniatis/Sambrook (Sambrook, J. et al. Molecular cloning: a laboratory manual. ISBN 0-87969-309-6) in order to reduce a-specific background signal.

The expression library was screened by plaque lift using an initial screening of approximately 20.000 plaques. The procedure was done as described in the manufacturers handbook (Clontech, Palo Alto, Calif., USA). All library screenings were done under native conditions. In short, phage-infected Escherichia coli XL1 Blue cells were plated in LB top agar onto LB agar plates both supplemented with 10 mM MgSO₄. The plates were then incubated at 42° C. for 4 hours. A nitrocellulose filter disc (Schleicher and Schuell, Dassel, Germany), previously soaked in 10 mM IPTG, was placed on each plate in order to induce expression of the proteins encoded by the cloned Ornithobacterium rhinotracheale inserts. After 4 hours incubation at 37° C. all filters were removed from the plates. After washing and blocking, filters were incubated with chicken antiserum (pooled from 10 animals, 1:250 dilution). The antiserum used in the first screening was obtained from chickens live vaccinated with Ornithobacterium rhinotracheale serotype G followed by a challenge with Ornithobacterium rhinotracheale serotype A. As secondary antibody rabbit anti-chicken IgG peroxidase (Nordic, Tilburg, The Netherlands) was use at 1:1000 dilution. As substrate solution Vector SG (Vector, Burlingame, Calif., USA) was used.

From the initial screening of 20.000 plaques, 200 reactive plaques were located on the agar plates and isolated. A plaque lift and screen as described above was repeated twice resulting in 175 single, pure reactive plaques. The pure clones were then spotted in duplo onto an E. coli XL1 Blue top agar lawn to give confluent plaques of approximately 5 mm diameter.

Again a plaque lift was performed and the filters were incubated with the antisera obtained from birds live vaccinated with Ornithobacterium rhinotracheale serotype B or serotype M prior to Ornithobacterium rhinotracheale serotype A challenge. Out of 175 reactive plaques, 30 plaques were selected to be cross-reactive with sera from birds live vaccinated with Ornithobacterium rhinotracheale serotype B, serotype G, or serotype M, and challenged with Ornithobacterium rhinotracheale serotype A.

### Example 2

### Identification of Open Reading Frames (ORFs) Encoding Antigenic Proteins and Expression in Escherichia coli

The DNA inserts of the 30 selected plaques were analysed in order to identify the open reading frames encoding the antigenic proteins. Oligonucleotide primers designed for the λTriplEx vector arms were used for both PCR amplification and sequencing. PCR was performed in a final reaction volume of 50 μl containing 50 M dNTP's (Promega, WI, USA), 10 pmol of both primers, 20 U/ml Supertaq plus polymerase and 10× Supertaq buffer (both HT Biotechnology Ltd, Cambridge, UK) in water. Phage DNA was added by picking a freshly plated plaque using a tooth pick, and transferring this DNA from tooth pick to reaction mix. The following conditions were used: denaturation at 94° C. for 3 min, followed by 30 cycles of denaturation at 94° C. for 1 min, annealing at 50° C. for 2 min and elongation at 68° C. for 2 min 30 sec, followed by a final extension at 68° C. for 10 min. To determine the nucleotide sequence of the amplified DNA inserts a sequence reaction was done (94° C. 10 sec; 50° C. 5 sec; 60° C. 2 min for 25 cycles) using Big dye Terminator Ready reaction mix (Qiagen Inc., CA, USA), 50 ng template DNA (PCR product) and 2.4 μmol primer in a 20 μl reaction volume.

After sequence analysis the 30 clones appeared to represent 8 different genes. Since most open reading frames where a fusion with the lacZ gene of the λTriplEx vector, the 5′ end of the gene was missing. For that reason a sequence reaction was performed using internal primers and chromosomal DNA of Ornithobacterium rhinotracheale serotype G as a template to sequence the missing 5′ gap.

Oligonucleotide primers were designed to amplify the full length open reading frames encoding the 8 cross-reactive antigens (Or01, Or02, Or03, Or04, Or11, Or77, Or98A and Or98B) from genomic DNA of Ornithobacterium rhinotracheale serotype G strain O-95029 nr.16279 (see table 1). The 5′ oligonucleotide primers contain a restriction site (underlined) preceding the ATG initiation codon (bold) followed by sequences derived from the gene of interest (italic). The 3′ oligonucleotides contain coding sequences (italic) followed by a restriction site (underlined). The PCR products were cloned in the expression vector of interest Ligation products were transformed to E. coli BL21 (DE3) codon RIL pLysS host cells Novagen, Madison, Wis., USA) for protein expression. By using the pET plasmid vector (pET22b) and a T7 RNA polymerase expression system (Novagen, Madison, Wis., USA), the recombinant proteins were expressed in E. coli, with an E. coli pelB leader peptide fused at the amino terminal portion (Ornithobacterium rhinotracheale leader peptides of proteins Or02, Or03, Or11, and Or77 were replaced) and 6 histidine residues at the carboxy terminal portion of the protein. E. coli strain BL21 (DE3) codon RIL pLysS Novagen, Madison, Wis. USA) was used for high level expression during IPTG-induction as described in the pET system manual (Novagen, Madison, Wis., USA).

### Example 3

### Purification of Antigens, Vaccine Formulations and Serological Analysis

Recombinant antigens expressed in E. coli were isolated from supernatant (Or77), purified by metal affinity chromatography using talon resin (Clontech Inc., Palo Alto, Calif., USA) as described by the manufacturer (Or03, Or04, Or98A and Or98B), or by repeated freeze-thawing, sonification, and centrifugation cycli (Or01, Or02 and Or11). Polyacrylamide gel electrophoresis (PAGE) followed by Coomassie brilliant blue staining was used to assess the purity of the recombinant proteins. Protein concentrations were estimated using bovine serum albumin as the standard.

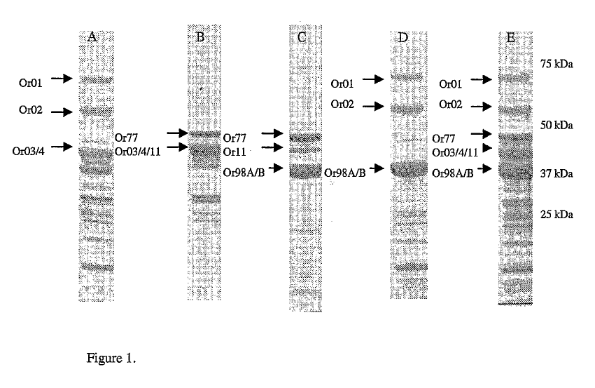

All purified recombinant proteins (Or01, Or02, Or03, Or04, Or11, Or77, Or98A and Or98B) were formulated individually in a water in oil emulsion. Furthermore, five different subunit vaccines (A, B, C, D and E) were formulated, containing different compositions of the 8 recombinant antigens (table 2). Coomassie staining of the 5 combination vaccines showed clearly identifiable protein bands corresponding to recombinant proteins Or01, Or02 and Or77. As the molecular weights of Or03, Or04 and Or11, and the molecular weights of Or98A and Or98B are approximately the same, individual protein bands could not be distinguished (FIG. 1). All proteins are present in approximately equal concentrations of 50 mg/antigen/l (25 μg/dose). Therefore, the total antigenic load of vaccine A to D is 200 mg/l. The antigen concentration of vaccine E is 400 mg/l. The protein background is rest material from E. coli strain used to express the recombinant Ornithobacterium rhinotracheale antigens.

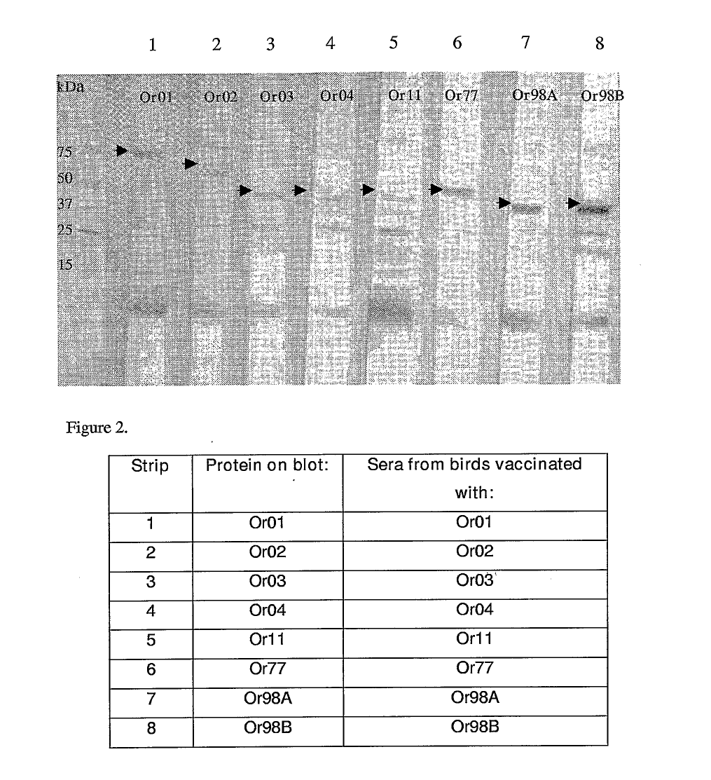

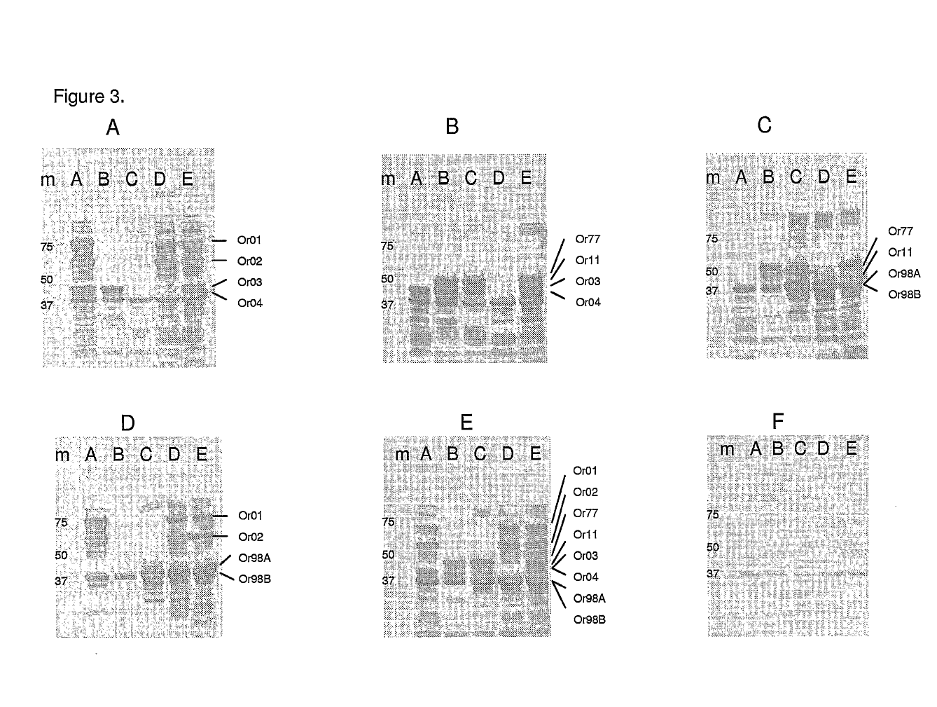

The ability of the different subunit vaccines to stimulate the humoral immune response to produce protein-specific antibodies was studied by subcutaneous injection of 2-weeks-old SPF-broiler chickens with 0.5 ml vaccine. Four weeks after vaccination serum-samples were collected and tested for the presence of antibodies reactive against the recombinant proteins. Semi-dry Western blotting was performed according to Towbin, H., Staehlin, T., and Gordon, J. (1979) Proc. Nat. Acad. Sci. 76:43-50. The protein phase of the vaccines was blotted and incubated with pooled serum (1:100 dilution) from vaccinated and unvaccinated birds. Sera obtained from birds vaccinated with each of the 8 individual vaccines Or01 to Or98B showed protein-specific reactivity (FIG. 2). FIG. 3 shows the reactivity of antisera obtained from birds vaccinated with subunit vaccine A to E (see table 2 and FIG. 1), directed against the same vaccines on Western blot. For example: blot A is loaded with vaccine A, B, C, D, and E (corresponding with lanes A to E). The serum used for primary antibody binding is obtained from birds vaccinated with vaccine A (corresponds with blot-number). For this reason, α-Or01, α-Or02, α-Or03 and α-Or04 antibodies are present in this serum. On blot A, these four proteins are stained in lane A, D, and E, which are the lanes that were loaded with the three vaccines that contain these antigens (A, D, and E). Blot B is loaded as blot A and the serum used is obtained from birds vaccinated with vaccine B. α-Or77, α-Or11, α-Or03, and α-Or04 antibodies stain the corresponding antigens on blot B in lane B, C, and E. The other antigens that were not present in vaccine B could not be detected on this blot. On blot E, all proteins are stained because vaccine E contains all eight Ornithobacterium rhinotracheale antigens. The serum used on Westernblot F is obtained from unvaccinated birds that served as a negative control. No recombinant Ornithobacterium rhinotracheale antigens could be detected using this serum.

### Example 4

### Protection Studies

To assess the cross-protective capacity of the antibody response induced by different subunit vaccines (combi vaccines A, B, C, D, E, and individual vaccine Or77), an animal experiment was performed. SPF-broilers were vaccinated at 2 weeks of age as described before. At 5 weeks of age birds were primed with ND LaSota (dose: 1*10⁶ E.I.D. per bird) by aerosol spraying. At 6 weeks of age, birds were challenged with Ornithobacterium rhinotracheale serotype A strain B3263/91 (heterologous challenge). The challenge was done by aerosol spraying of a fresh bacterial culture containing 8.5*10⁸ colony forming units (CFU) per ml THB. During aerosol challenge the bacterial culture was administered as a fine spray to the birds in an isolator of approximately 1.5 m³, using a commercial paint sprayer. The developed mist in the isolators was maintained for at least 10 min with the air circulation closed. Challenge control groups and ND priming groups were included in the test. One week after challenge, at 7 weeks of age, birds were sacrificed and organ lesions were macroscopically scored using an Ornithobacterium rhinotracheale scoring system for respiratory disease as follows: for thoracic air sacs, 0=no abnormalities, 1=one air sac seriously affected by fibrinous airsacculitis or limited pin-head sized foci of fibrinous exudates in both air sacs, 2=both air sacs seriously affected by fibrinous airsacculitis; for abdominal air sacs, 0=no abnormalities, 1=pin-head sized foci of fibrinous exudates or slight diffuse fibrinous airsacculitis, 2=severe fibrinous airsacculitis. The airsacculitis score is given as the sum of both scores. For lungs, 0=no abnormalities, 1=unilateral pneumonia, 2=bilateral pneumonia. The average group scores are given as a percentage of the maximum possible score. Statistical analysis was performed using Kruskal-Wallis non-parametric one-way ANOVA.

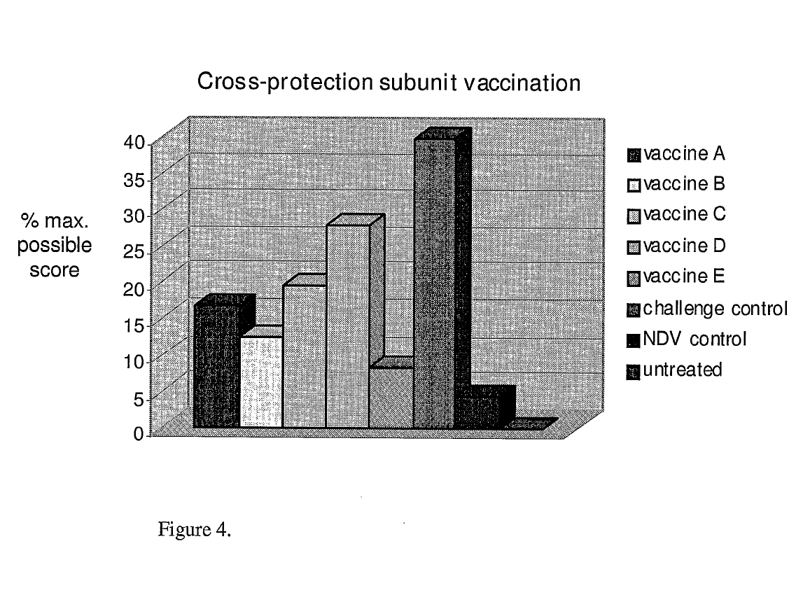

FIG. 4 shows the cross-protective capacity of the 5 different subunit vaccines A to E. The challenge control group was not vaccinated but primed and challenged and showed the highest score. Birds vaccinated with vaccine E (containing all 8 antigens) showed almost complete protection comparable to the results of the group that did not receive vaccination and challenge but was primed with Newcastle Disease virus. A somewhat lesser, but still significant cross-protection (P<0.05) could be observed in birds vaccinated with vaccine A, B and C. Combination vaccine D showed cross-protection of less significance (p=0.19). Untreated birds showed no organ lesions.

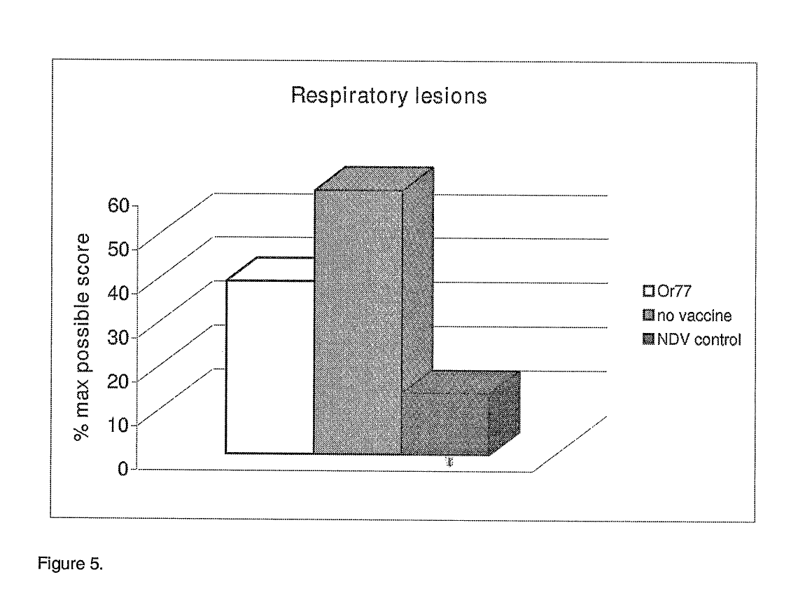

As can be seen from FIG. 5, the Or77 (=serotype G strain)-vaccinated and serotype A challenged animals also show a significant (p<0.05)) reduction in respiratory lesion scores compared to the unvaccinated control group.

Legend to the figures:

FIG. 1: Coomassie staining of the 5 combination vaccines (A to E). Each vaccine containing a different composition of the 8 purified recombinant proteins. Subunit vaccine A corresponds with lane A, subunit vaccine B corresponds with lane B, subunit vaccine C corresponds with lane C, subunit vaccine D corresponds with lane D, subunit vaccine E corresponds with lane E. Recombinant proteins with approximately equal molecular weights are indicated by a single arrow.

FIG. 2: Reactivity of monovalent antisera, obtained from chickens vaccinated with the single recombinant subunit vaccines, against the same protein on Western blot. The reactive vaccine proteins are indicated with black arrows.

FIG. 3: Reactivity of antisera, obtained from chickens vaccinated with subunit vaccines A to E on Western blot. Each blot contains the proteins of vaccine A, B, C, D, and E (corresponding to lanes A to E). The serum used for screening is obtained from birds vaccinated with vaccine A (blot A), vaccine B (blot B), vaccine C (blot C), vaccine D (blot D) or vaccine E (blot E). The serum used on Western blot F is obtained from unvaccinated birds. The reactive vaccine proteins are indicated with a black line.

FIG. 4: Cross-protective capacity of subunit vaccines A to E, in comparison to challenge and NDV control groups, represented as the maximum possible respiratory organ lesion score.

FIG. 5: Cross-protective capacity of subunit vaccine Or77, in comparison to challenge and NDV control groups, represented as the maximum possible respiratory organ lesion score.

TABLE 1

Oligonucleotide sets used for cloning selected Ornithobacterium rhinotracheale genes encoding
cross-reactive antigens

| | | Restriction | | Restriction |
|---|---|---|---|---|
| Gene | 5′oligonucleotide | site | 3′oligonucleotide | site |
| Or01 | 5′-GCTGGCCATGGCTGAAATTATAAAAATGCC-3′ | MscI | 5′-CCGCTCGAGCACAAGCATAGACATTGG-3′ | XhoI |
| Or02 | 5′-CAGTCCATGGCATGTAGCGATTTTGAT-3′ | NcoI | 5′-CCGCTCGAGGTGGTCTTTATAAAAATG-3′ | XhoI |
| Or03 | 5′-CAGTCCATGGCGATGATAATCAGTTCTTATG-3′ | NcoI | 5′-CCGCTCGAGAATAAATTCATCATTAAGC-3′ | XhoI |
| Or04 | 5′-CGATGGCCATGAAAGATATATTTGAAT-3′ | MscI | 5′-CCGCTCGAGTTCTTCACTTGGTATTTTGA-3′ | XhoI |
| Or11 | 5′-CGATGGCCATGGGGGCACAAGGTGTAGC-3′ | MscI | 5′-GCGGCCGCTACGATAAACCTAGACCAAA-3′ | NotI |
| Or77 | 5′-CATGCCATGGTCTGTAGCAGTGATGATTAC-3′ | NcoI | 5′-CCGCTCGAGGTTAATTGAAACTCTTAAGC-3′ | XhoI |
| Or98A | 5′-CAGTCCATGGTAAAAGACTTTTCAG-3′ | NcoI | 5′-CCGCTCGAGTGCTATTAATTCTAATCG-3′ | XhoI |
| Or98B | 5′-CAGTCCATGGAATTAGCGAAAAACGAC-3′ | NcoI | 5′-CCGCTCGAGTTTTAATTCATTTTTTCTG-3′ | XhoI |

Restriction site: underlined
ATG start codon: bold
Gene of interest: italic

TABLE 2
Subunit vaccines (A to E) consisting of different protein subset combinations

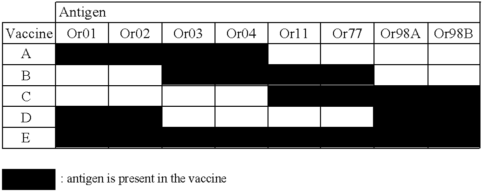

1-52. (canceled)

53. An isolated Ornithobacterium rhinotracheale protein or an isolated immunogenic fragment thereof, wherein said protein or said immunogenic fragment induce both a protective homologous immune response and a protective cross-reactive immune response.

54. The protein or immunogenic fragment of claim 53, wherein said protein or immunogenic fragment is selected from the group consisting of SEQ ID NO: 2, SEQ ID NO: 4, SEQ ID NO: 6, SEQ ID NO: 8, SEQ ID NO: 10, SEQ ID NO: 12, SEQ ID NO: 14 and SEQ ID NO: 16.

55. An isolated nucleic acid molecule encoding the protein or immunogenic fragment of claim 54.

56. The nucleic acid molecule according to claim 55, wherein said nucleic acid molecule is selected from the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, and SEQ ID NO: 15.

57. The nucleic acid molecule according to claim 55, wherein said nucleic acid molecule hybridizes under stringent conditions to a member of the group consisting of SEQ ID NO: 1, SEQ ID NO: 3, SEQ ID NO: 5, SEQ ID NO: 7, SEQ ID NO: 9, SEQ ID NO: 11, SEQ ID NO: 13, and SEQ ID NO: 15.

58. A DNA fragment comprising the nucleic acid according to claim 55.

59. A recombinant DNA molecule comprising the nucleic acid according to claim 55 under the control of a functionally linked promoter.

60. A live recombinant carrier comprising the nucleic acid according to claim 55.

61. A host cell comprising the nucleic acid according to claim 55.

62. A vaccine for combating Ornithobacterium rhinotracheale infection, wherein said vaccine comprises the protein or fragment of claim 53, and a pharmaceutically acceptable carrier.

63. The vaccine of claim 62, further comprising an adjuvant.

64. The vaccine according to claim 62, wherein said vaccine comprises an additional antigen derived from a virus or micro-organism pathogenic to poultry, an antibody against such an antigen or genetic information encoding said antigen.

65. the vaccine according to claim 64, wherein said virus or micro-organism pathogenic to chickens is selected from the group consisting of Fowlpox virus, Infectious Bronchitis virus, Infectious Bursal Disease (Gumboro), Marek's Disease Virus, Chicken Anaemia agent, Avian Reovirus, Mycoplasma gallisepticum, Turkey Rhinotracheitis virus, Haemophilus paragallinarum (Coryza), Chicken Poxvirus, Avian Encephalomyelitisvirus, Duck Plague virus, Newcastle Disease virus, Egg prop syndrome virus, Infectious Laryngotracheitis virus, Herpes Virus of Turkeys, Eimeria species, Ornithobacterium rhinotracheale, Pasteurella multocida, Mycoplasma synoviae, Salmonella species and E. coli.

66. A vaccine for combating Ornithobacterium rhinotracheale infection, wherein said vaccine comprises the nucleic acid molecule of claim 55, and a pharmaceutically acceptable carrier.

67. The vaccine of claim 66, further comprising an adjuvant.

68. A vaccine for combating Ornithobacterium rhinotracheale infection, wherein said vaccine comprises antibodies against the protein or fragment of claim 53, and a pharmaceutically acceptable carrier.

69. The vaccine of claim 68, further comprising an adjuvant.


SEQUENCE LISTING (16 sequences)

SEQ ID NO: 1 - DNA, 1614 bp, Ornithobacterium rhinotracheale
  CDS (1)..(1614)
atggctgaaattataaaaatgccaagattgagcgataccatggaagaaggtaaagtggaatcttggaacaaaaaagtaggagataaagtatcatacggcgacatcttagccgaaatcgaaacagataaagcggttcaagaatttgaaacagatgtagaaggtactcttttatacatcggtgtagaggctggtcaagcagcaccagttgatagtattttagctatcatcggtgcagaaggcgaagacatcagcggtttggtaagcggtggaggtgctagccaatcagcgccagctcaagaagctgccgctcctgcagaagaaccacaagcggaagctgcaccagcggctgaagttccagaaaatgtaactatcgtttctatgccaagattgagcgataccatggaagaaggtaaagtagaatcttggaacaaaaaagtaggagataaagtatcatacggcgacatcttagccgaaatcgaaacagataaagcggttcaagaatttgaaacagatgtagaaggtactttattatatataggtgtagaagctgggcaatcagcaccagttgatagcattttggcaatcatcggacctgaaggaacagatgtttctgcaatcgtagcaggaggtggtgcaaaaccagctgctaaagcggaagctccaaaggctgaagcacctaagcaagctgctccagcacaagagaaaaaagaaactccagcgcctgctgctccaaaagcacaagctaccaacaattcaggtagagtatttatttctccattggctaaaaaattggctgatgaaaaaggatacgatatcaatcaaattcaaggtacaggagacaacggaagaatcatcaaaaaagatgttgaaaactttactccacaagctgctgcggctaagccagctgttgctggtccagttgcattggaagtaggagaagatactgtaatccctaactctcaaatgagaaaagtgattgctaagcgtctttctgaaagtaaatttacagcaccacactactacttaaccattgaagtagatatggataatgtgatggcggctcgtaagcaaatcaaccaaattccaaatacaaaagtatctttcaacgatatcgtattgaaggctactgctatggctgtgaaaaaacacccagtggtaaattcaacttggaaagataacgaaatcgtacaatacgctgctgtaaacatcggtgttgcagttgctgttccagatgggcttgtagtacctgtagtgaaaaatacagatttaaaatcattatctcaaatttctgctgaggtaaaagatttagctacaagatcaagagatagaaaaatcaaagctgatgagatggaaggttctacctttacagtttctaacctaggagcttacggtgtagaaagctttacatcaatcatcaatcagccaaactcttgtatcctttctgtaggtgcgattgtagaaaaaccagttgttaaaaacggacaaatcgtagttggtcacacaatgaaactttgtttagcttgcgatcacagaactgtggacggagcaactggaagtactttcctacaaactttaaaacaatacttagagactccaatgtctatgcttgtgtag

SEQ ID NO: 2 - protein, 537 aa, Ornithobacterium rhinotracheale
MAEIIKMPRLSDTMEEGKVESWNKKVGDKVSYGDILAEIETDKAVQEFETDVEGTLLYIGVEAGQAAPVDSILAIIGAEGEDISGLVSGGGASQSAPAQEAAAPAEEPQAEAAPAAEVPENVTIVSMPRLSDTMEEGKVESWNKKVGDKVSYGDILAEIETDKAVQEFETDVEGTLLYIGVEAGQSAPVDSILAIIGPEGTDVSAIVAGGGAKPAAKAEAPKAEAPKQAAPAQEKKETPAPAAPKAQATNNSGRVFISPLAKKLADEKGYDINQIQGTGDNGRIIKKDVENFTPQAAAAKPAVAGPVALEVGEDTVIPNSQMRKVIAKRLSESKFTAPHYYLTIEVDMDNVMAARKQINQIPNTKVSFNDIVLKATAMAVKKHPVVNSTWKDNEIVQYAAVNIGVAVAVPDGLVVPVVKNTDLKSLSQISAEVKDLATRSRDRKIKADEMEGSTFTVSNLGAYGVESFTSIINQPNSCILSVGAIVEKPVVKNGQIVVGHTMKLCLACDHRTVDGATGSTFLQTLKQYLETPMSMLV

SEQ ID NO: 3 - DNA, 1572 bp, Ornithobacterium rhinotracheale
  CDS (1)..(1572)
atgaaaataaattacaaaaatatacttttaagtgctagcgttctcttttttgcagcatgtagcgattttgattacaatgtagaaaacccaaacctcacgaagggagaggctgatttctctaaatatgtagctttaggaaattctctcacttctggttattcagacggagccttatatcgctcggcacaagagaattcataccccgcaatcattgccaaacaaatgaaatatgtaggcggtggcgagttctctcaacctttgatgaaagacaacattggtggtttttcggatttgtttgaagcaagtaaacacaccgcattttacggaaaattagaattaaaaatcgtagacggtgcacctacgccagtgccttctgtgcctaagtttagtttagctcaaaccttcgtaaaagggaattttaataatttgggcgtgccaggggctaaatcttatcatttattagctcaaggttacggaaatattgctaatctgaaggagagtaaagccaatccatattttgtgcgatttgctagccaaccaaatgccagcgtgctgagcgatgctttggcacaaaaacctacattctttaccttatggatcgggaacaacgatgttttaggctatgccatgaatggcgcagcaagcacagatcgaaaagggaaccctgatgtaacaacatataattcaaatgatttgtctgatgctaacttggtggcaggctctattcaaaaattagtaaaagcacttacagattcaggcgcaaaaggtgctgtagcgaatttgccttatgtcgaagacattccgtattttacaaccgtgccggctgagcctttaagccctttaaacaaaagttacgctacacaaattgaaaatttgaataaattttatgctagcctaaataaagtttttgatgccctaggagcaagcgatagaaaaatcacatttaatgccgataaagcaagcggtgctgtgattgtagataaaagtttgccagatttaagtcaaaaaatcttagcaaccttactaaaattagaattcccaaacgaaaaagctaaacttttagcacaaacctttggtcaagtgcgccaatctaaagcaggagatttattgccacttacagcgagtagaacacttgggaaattaaatagtgaaagacttgctactttgacaaaattaggattaccaaaggaaaacgccgctcaactttctatgaacggacttacttatccattgcaagatgccgatgttttaaccaaaaatgaagtttcaacaattcacgaaagagtaaacgaaatcaatcaaggcatacaagcagtggcaaaacaattcaacattgcatatgtggacatgaatgccgaaatgcaaaaactcactaaaggctttaaattcaacggggtagactacaacgcaagttttgtgactggtggagctttttcgcttgatggagtgcatttaaacagccgaggatatgcccatacagctaatacatttattcgtgccatcaatcagcaatataaggcaagcattccgttggtagatatcaacgctttcccaggcacacaattaccttaa

SEQ ID NO: 4 - protein, 523 aa, Ornithobacterium rhinotracheale
MKINYKNILLSASVLFFAACSDFDYNVENPNLTKGEADFSKYVALGNSLTSGYSDGALYRSAQENSYPAIIAKQMKYVGGGEFSQPLMKDNIGGFSDLFEASKHTAFYGKLELKIVDGAPTPVPSVPKFSLAQTFVKGNFNNLGVPGAKSYHLLAQGYGNIANLKESKANPYFVRFASQPNASVLSDALAQKPTFFTLWIGNNDVLGYAMNGAASTDRKGNPDVTTYNSNDLSDANLVAGSIQKLVKALTDSGAKGAVANLPYVEDIPYFTTVPAEPLSPLNKSYATQIENLNKFYASLNKVFDALGASDRKITFNADKASGAVIVDKSLPDLSQKILATLLKLEFPNEKAKLLAQTFGQVRQSKAGDLLPLTASRTLGKLNSERLATLTKLGLPKENAAQLSMNGLTYPLQDADVLTKNEVSTIHERVNEINQGIQAVAKQFNIAYVDMNAEMQKLTKGFKFNGVDYNASFVTGGAFSLDGVHLNSRGYAHTANTFIRAINQQYKASIPLVDINAFPGTQLP

SEQ ID NO: 5 - DNA, 1242 bp, Ornithobacterium rhinotracheale
  CDS (1)..(1242)
atgaaaaaactatcatatttattattctcaattccactattgtggggattgaatgcttgtaccgaagattttgaacccacattttcagaaaacgcaactcagcgttatataaatgttcagaacgaaattacagaatttcttagtacccctgatgctgattttattttacaatacttcccagatgataatcagtcttatggaggatataattatttcttgaaattctcaggaaaagataaggtaagtgcggaatcagagaccaatgaacaagctgtaagttctacttttcgaattcttcaaaatggaggtgcagttcttacctttgatttatacaatgaggagctacatgaatttgcaactcctagtccatcagaatatcgtgcaaaacgaggagattttgaatttttgatccttaaaaaaagtaatgacacactttatctaaaaggaaagaaaacaggaaattacatgaagctatataaagcagggaatattcaagagattaaaagtaacattagaaaagtagcaactacaattgatagggtagatcttccagctcaaggtactataggtacagagcctttggtattgtcaacaggaggaactagaaatattatttttagtactttaaatggggggagtatagagtctacagaagcatcgtatatttttacagaaaacggaattaagttttacaaaccagttgaaattaaggggaaagtttacggtggattaatttttgacgaaagtactcaaacattaaagtcagaagatggtgtaattgtaattaatttgaaatttgttcctatcaactttaaatcaaaagcttggtttttggatatgagcaaatcagagaatacatcggaaggttataagaaagccagagcaggcgatagtcttttgcatggtatgattctaagtaaatttaagttacaagatttctatgtgttaggtaattttagagataatgtaggattcaatacttttgttgagggctataacggagcatttgcaatttatggtttaagtttcaaaggagaagattcaaatccaaatcttatccacattgagaaaacaaaacctgttgaatttgatgcttatttcaaatatgtgaatggagttttagataaaatcactaaaaattcaccttatattgtagaggaggttcagtcagatcctaaacgtgtgaagctaataagtaaaaatgatcaagaattatggtttattcttgatttgcttaaatga

SEQ ID NO: 6 - protein, 413 aa, Ornithobacterium rhinotracheale
MKKLSYLLFSIPLLWGLNACTEDFEPTFSENATQRYINVQNEITEFLSTPDADFILQYFPDDNQSYGGYNYFLKFSGKDKVSAESETNEQAVSSTFRILQNGGAVLTFDLYNEELHEFATPSPSEYRAKRGDFEFLILKKSNDTLYLKGKKTGNYMKLYKAGNIQEIKSNIRKVATTIDRVDLPAQGTIGTEPLVLSTGGTRNIIFSTLNGGSIESTEASYIFTENGIKFYKPVEIKGKVYGGLIFDESTQTLKSEDGVIVINLKFVPINFKSKAWFLDMSKSENTSEGYKKARAGDSLLHGMILSKFKLQDFYVLGNFRDNVGFNTFVEGYNGAFAIYGLSFKGEDSNPNLIHIEKTKPVEFDAYFKYVNGVLDKITKNSPYIVEEVQSDPKRVKLISKNDQELWFILDLLK

SEQ ID NO: 7 - DNA, 1023 bp, Ornithobacterium rhinotracheale
  CDS (1)..(1023)
atgaaagatatatttgaatatacacttctagcattaggtggtttgctactcaccaattgctatgatagcgatgagattgaagtaattaaatttgatgattcttttactccagctccgcccaccgaaaaaaaaagagacactccgctaataaatttattagatgattttgtattctttaaaaaagatgtagtaacaattccggtagataaagacaatttagccaccaataatgtcatcagtggtgaagtctttacaaatagaaaaatgtctgaaaattttgagtatcagcttgaattagaccaagattggattagtagcaatccggacttacaagccattccaaacggagcttttacaatctctggacaaacactcaacaaagatgaaagaaatggtactttcaaaattcagcttaatgcagaggtggcgaaagagctaggaggcacctactatctcccgctaaaattggtttctaaaaatgataatttaaacattttaaagggatatgaaagtggcgtttttaagctagtattcaaaaaatcgtatccaatcccagaaggtaacaatgttgaaggaaaaaaaggatattattttgatggtttaggcaataatatacctagaacagatttatcgtttaattcaaattacgcccccgatcatctttttaaattaaatgatggaaaccaacaaggggctaattggtgggcagacactgatgataacacaacatatcttgatgtaaaattccctattaatacaataaaagctataaaattatacactaaaagctattggcaaaatgctgtaggcagtgtaaaaattgaagtttctaatgataatggcaatacttggaaagaacagggaattgctaactttgggcaatattcaacagtgtctactattgtattcactcaaccaattgacattaatgctgtcagaatatctaacttcactagagggggaagtagtaatttcattaacattaacgaggtggaagtattcaaaataccaagtgaagaataa

SEQ ID NO: 8 - protein, 340 aa, Ornithobacterium rhinotracheale
MKDIFEYTLLALGGLLLTNCYDSDEIEVIKFDDSFTPAPPTEKKRDTPLINLLDDFVFFKKDVVTIPVDKDNLATNNVISGEVFTNRKMSENFEYQLELDQDWISSNPDLQAIPNGAFTISGQTLNKDERNGTFKIQLNAEVAKELGGTYYLPLKLVSKNDNLNILKGYESGVFKLVFKKSYPIPEGNNVEGKKGYYFDGLGNNIPRTDLSFNSNYAPDHLFKLNDGNQQGANWWADTDDNTTYLDVKFPINTIKAIKLYTKSYWQNAVGSVKIEVSNDNGNTWKEQGIANFGQYSTVSTIVFTQPIDINAVRISNFTRGGSSNFININEVEVFKIPSEE

SEQ ID NO: 9 - DNA, 1230 bp, Ornithobacterium rhinotracheale
  CDS (1)..(1230)
atgattaaaaaagtatttttatcgtttgtgctcatggcaagcacaggcattttatgggcaggcggataccgagtttcgctgcaaggtgtaagacaagccgccatgggggcacaaggtgtagcactttctcacgatgcgagtgtggcatttttcaaccccgcagcattggcttttgtagatgataaattaagtattgctgtgggaggtttcggaattgggattaccgcaaaataccaaaaccgcgaaacgctctataaagccgaaaccgacaatccgctggggacaccactttatcttgctacaagctataagcctacggaaaaactagccttaggcgtgagcgtaaccactccgtttgggagcaccgtagactggggagataaatgggctggacgctacatcattgatagaattgccctcaaatcgttttttattcagcccacggcagcgtataaagtaaccgattggctctctgtgggggctggtgccatcatcgctcgaggcaatgtaaacattaagcgtgcaatatctctaggcaaccaagatgcggggctagaaatcgacaaaaaaggagctcacggaacagggtttaatgtaggggtttatgccaaaccaaatgataaattaaatataggaattgcttaccgatcagaagtgaagatgaaagcggacaaaggtgatgctgttttcaaaaatttaccaagtatcgtaaagggcaaaatgcctttttcggctaaatattttgatgctcaattacctctaccagcagaacttttaattggggcgaactataaagtaacaccaaaattgctcgtaggggcagaaattggggctgtaaaatggaacgcctacgaaacattaaatattaaactttataacaacgaagaggaatacaacaatacttctaacaaaaattacaaaaacacattaaattatagtatcggggctgaatatttaatcaatccaaaagctgccttacgcttagggtataaattcgacaaatcgccttcgccagctgattcgtttaacccagagaccccaaccattaattatcacgcatttacaactggatttggatatgaattcgagagatttcgtgtagatgccatggcggaatatttactaggaaacgaaagaagcttccacaatacacaatataactttgggggcgacatcaacactggtggctatgtgtttggtctaggtttatcgtatagacttgacaaataa

SEQ ID NO: 10 - protein, 409 aa, Ornithobacterium rhinotracheale
MIKKVFLSFVLMASTGILWAGGYRVSLQGVRQAAMGAQGVALSHDASVAFFNPAALAFVDDKLSIAVGGFGIGITAKYQNRETLYKAETDNPLGTPLYLATSYKPTEKLALGVSVTTPFGSTVDWGDKWAGRYIIDRIALKSFFIQPTAAYKVTDWLSVGAGAIIARGNVNIKRAISLGNQDAGLEIDKKGAHGTGFNVGVYAKPNDKLNIGIAYRSEVKMKADKGDAVFKNLPSIVKGKMPFSAKYFDAQLPLPAELLIGANYKVTPKLLVGAEIGAVKWNAYETLNIKLYNNEEEYNNTSNKNYKNTLNYSIGAEYLINPKAALRLGYKFDKSPSPADSFNPETPTINYHAFTTGFGYEFERFRVDAMAEYLLGNERSFHNTQYNFGGDINTGGYVFGLGLSYRLDK

SEQ ID NO: 11 - DNA, 1140 bp, Ornithobacterium rhinotracheale
  CDS (1)..(1140)
atgaagaaaatacttttagcaattagcttttcgtcttttgttttaagctgtagcagtgatgattacactccagccacacctaaagaaacagaaaagcctaaggaagaggctgtggttccaaataagccagatgaaccaaaggctgatgatggaaacgaaaatccagaaaacactggagatgaagagaatggagataatacaaactccgttgtcgggaagcctgatgatttccacatggggaatcgctcttatgctagctggaaagaagatgtggattatatcggaggttttgatattgaaactcttttaagtggggctgataatcaaaaatatgatgcggcttattttagccaatttatcaagatattctcatctagtccaaacggaaacaatttctacacttttcaggcagaagactttaaagatgtcgagattaaagacttaaagtttgatattggtagaaatgtaattacatttaaaactagctacaaaggcgtaaaaagtgaaattacatcttctttaaaatttgatttggctaatttttatgatcgaaaaatcaaaataaacgaagatttcgttgcatctcactacatgagagggatttatgaggagcttggaggttttatcgggaatttattaaactacgacgatgagaaatacaatctagagttagcggggtcaaaaaacaaagatgaatccaataactctttaggttttagcattcgcgtaacagataaaaaagataagtatataacaacggtttataaaaacatctcaggatttaggcctctttctagtctgcaggaggagctttccattgctcctacttacgaattgcgagagaaaatcaaggagaaaatagatagaaataaaagaaacattagcctattggagctattaaaaccatcggtaaacgaatggatgaagtctgccgatttctactttaataacactgatttggaatggagaggagatcattattcagctagagggtttttagatttgtatataggttcgcctagatttgagctgattttagcaacaaaagaagacaattggttgattttgaaagtgaaagtggttcagataaatgaagtgcctaccgatttggtgtatagcttaagagtttcaattaactaa

SEQ ID NO: 12 - protein, 379 aa, Ornithobacterium rhinotracheale
MKKILLAISFSSFVLSCSSDDYTPATPKETEKPKEEAVVPNKPDEPKADDGNENPENTGDEENGDNTNSVVGKPDDFHMGNRSYASWKEDVDYIGGFDIETLLSGADNQKYDAAYFSQFIKIFSSSPNGNNFYTFQAEDFKDVEIKDLKFDIGRNVITFKTSYKGVKSEITSSLKFDLANFYDRKIKINEDFVASHYMRGIYEELGGFIGNLLNYDDEKYNLELAGSKNKDESNNSLGFSIRVTDKKDKYITTVYKNISGFRPLSSLQEELSIAPTYELREKIKEKIDRNKRNISLLELLKPSVNEWMKSADFYFNNTDLEWRGDHYSARGFLDLYIGSPRFELILATKEDNWLILKVKVVQINEVPTDLVYSLRVSIN

SEQ ID NO: 13 - DNA, 918 bp, Ornithobacterium rhinotracheale
  CDS (1)..(918)
atgattgtaaaagacttttcagactatacattccgatgttctcaattaggtaagttaatggttggtgtcaagccaccattaacccctaatcaagagaagttgctcacagacttagagggcaaaatggaagctgggaccattaccaaaaagcaaatcatcacttatggtgaattgctttccaagaaaaaccaaaagcttgaattatctgcaagtgtaaagtcttacttagccgacattcataaagaagtcttttttggtcgtgataaggaattgaccaataaatatctatcaaaaggcattcaagtagaagaaaagagcataacgctctattccgatgtctgtaacaagttattcctaaagaataaaaagttttacaaaaacgattttattcaaggtacgccagataacacgcaagacaaaatcagagatatcaaaagtagttgggacttctcaacctttcctctacacgccgatgaaacgccaaccaaagactatgaatggcagttgcaaggttatatggaattaacaggcttaaaagaagctgagttgatttattgcttggttgatacgcctcataaaattgtagaagatgaaatccgaagaatggactggaagcataatttacttgacattaacggcgaagtgagagccgagacaagagatttagtagttgagattgtgtctaacttaatttataccaagcaaggcttggaagacttttgtcagcagtccgcagtcataaacaaagattggttcacggactttgaggaaataccacaagaattgagaattaaagtttttcactttgagcatcaaaaagagatgattagcgcactctacgagcaaataggaagatgtagagcgcatttaaacgacttgaccatgaaaatggcaacacgattagaattaatagcataa

SEQ ID NO: 14 - protein, 305 aa, Ornithobacterium rhinotracheale
MIVKDFSDYTFRCSQLGKLMVGVKPPLTPNQEKLLTDLEGKMEAGTITKKQIITYGELLSKKNQKLELSASVKSYLADIHKEVFFGRDKELTNKYLSKGIQVEEKSITLYSDVCNKLFLKNKKFYKNDFIQGTPDNTQDKIRDIKSSWDFSTFPLHADETPTKDYEWQLQGYMELTGLKEAELIYCLVDTPHKIVEDEIRRMDWKHNLLDINGEVRAETRDLVVEIVSNLIYTKQGLEDFCQQSAVINKDWFTDFEEIPQELRIKVFHFEHQKEMISALYEQIGRCRAHLNDLTMKMATRLELIA

SEQ ID NO: 15 - DNA, 888 bp, Ornithobacterium rhinotracheale
  CDS (1)..(888)
atgaacgaattagcgaaaaacgacatcaagtcattgttaaaaagtgccgacatcaacaaaagatttgagcaattgctcggcaaaaaagcacaaggctttatctcatcagtcttgcagacggcacaaaataacagattgttagcgacagccgacccaaagaccattctaaacgctgcagtaacagccgcgactttagatttgccaattaatcagaatttaggttacgcctacatcgtgccttacaaagggcaggcgcaattccaattaggctggaagggctttgtagcattagctaaaagaagtggcgcatatttgaaaatgaatgtagtaactgtctatcaaaatcaattcaaatcctacaatcgcttaacagaagaattagatgccgatttcacaatcgaaggcaatggtgaagtagttggttatgcagcctatttcaaagaaatcaatggttttgaaaagctttcgttttggtcaattgagcaagtaaaaaaacacgccaccaaatactctcaaacttatggtaaaaaatcacgctcgggggcattaatgttttcgccttggaatgatgaagaccagtttgacgcaatggctatgaagactgtcttaaaaaacacgctctcaaagtttgggacactctcaattgaaatgcaaatggcgcaaatggcagaccaagcagtcatcaagaacgagggggagtacgagtatatagacaataccatagacattgaagctgaaagtgccgaagaagaagccaatcgtattatgaaatttattgataaagccgaaagcattgaagccttagaggaattaaaatcatcagttgatgagaatggcgatttagagttattagcctattacgacaacagaaaaaatgaattaaaatga

SEQ ID NO: 16 - protein, 295 aa, Ornithobacterium rhinotracheale
MNELAKNDIKSLLKSADINKRFEQLLGKKAQGFISSVLQTAQNNRLLATADPKTILNAAVTAATLDLPINQNLGYAYIVPYKGQAQFQLGWKGFVALAKRSGAYLKMNVVTVYQNQFKSYNRLTEELDADFTIEGNGEVVGYAAYFKEINGFEKLSFWSIEQVKKHATKYSQTYGKKSRSGALMFSPWNDEDQFDAMAMKTVLKNTLSKFGTLSIEMQMAQMADQAVIKNEGEYEYIDNTIDIEAESAEEEANRIMKFIDKAESIEALEELKSSVDENGDLELLAYYDNRKNELK